(19) United States
(12) Patent Application Publication
(10) Pub. No.: US 2024/0038368 A1
(43) Pub. Date: Feb. 1, 2024

(54) SYSTEMS AND METHODS TO ELIMINATE NOISE FROM IMAGING DATA

(71) Applicant: GE Precision Healthcare LLC, Waukesha, WI (US)

(72) Inventors: Harsh Agarwal, Bangalore (IN); Ramesh Venkatesan, Bangalore (IN); Ravi Jaiswal, Bangalore (IN); Santosh Kumar, Bangalore (IN)

(21) Appl. No.: 18/358,326

(22) Filed: Jul. 25, 2023

(30) Foreign Application Priority Data
Jul. 28, 2022 (IN) 202241043290

(51) Int. Cl. G16H 30/40; G06T 7/00

(52) U.S. Cl. CPC G16H 30/40; G06T 7/0012; G06T 2207/10088; G06T 2207/20182

(57) ABSTRACT

The disclosure relates to a system and a method to eliminate noise from imaging data The disclosure provides system comprising a plurality of coils configured to generate one or more data sets and a processor may be configured to receive the data sets from the plurality of coils. The processor may determine an interference removal parameter from the data sets received from the plurality of coils. The interference removal parameter may remove noise from the one or more data sets.

### CROSS REFERENCE

The present application claims priority and benefit of Indian Patent Application No. 202241043290 filed on Jul. 28, 2022, which is incorporated herein by reference in its entirety.

### FIELD OF THE DISCLOSURE

This disclosure relates generally to an improved imaging system and, more particularly, to improved system and method to eliminate noise due to radio interference in a Magnetic Resonance Imaging (MRI) system.

### BACKGROUND

Magnetic Resonance Imaging (henceforth referred to as MRI) is one of the primary medical imaging technologies. The principle of MRI is to obtain electromagnetic signals from the human body by utilizing the magnetic resonance phenomenon. The human body majorly comprises water. Each molecule of the water has two hydrogen atoms and for imaging purpose the hydrogen proton may be used for generating the image. A hydrogen proton is akin to the planet earth and the magnetic spin on its axis. The hydrogen proton has a south and a north pole. The hydrogen proton behaves as a bar magnet in presence of magnetic field.

A hydrogen proton has two energy levels under a static magnetic field, and when excited at radiofrequency (henceforth referred to as RF), the hydrogen proton transit from a lower energy level to higher energy level. If an RF pulse is removed, the hydrogen proton transits from the higher energy level back to lower energy level and emit RF energy. This RF energy may be received by the RF coils. MRI produces non-invasive and high-resolution images of internal organs/structures within the human body for diagnostics, therapeutic and/or research purpose.

The MRI systems are designed to pick up a weak MRI signal. However, the sensitive antennas/RF coil may be susceptible to undesirable external radio frequency signals in form of noise signals, which may be received by a receive coil. The noise signal may appear as artifacts in images as zipper artifacts running across an imaging data. The presence of such artifacts may cause hindrance in proper clinical diagnosis of a subject.

Existing techniques provide several measures to eliminate the radio frequency interference. One of the known techniques provides an MRI system installed in a room shielding structure having thick copper plates and then notch filters are used for any cable entering or exiting the room where the MRI system is disposed. The size of the entire system, along with the room shielding, is considerably demanding and requires separate room for both the MRI systems and MRI control units, which may be used for controlling the MRI system. This limitation automatically restricts the implementation and usage of the MRI system in a large scale and creates several restrictions in terms of installation, servicing, and cost.

Another known technique provides auxiliary coils, which may be capable of receiving noise signal from one or more noise sources present in the surrounding environment in which the MRI system may be operating and then measuring the noise. The auxiliary coil measures the noise from one or more noise sources in the environment in which the MRI system operates. The use of auxiliary coil required additional hardware to receive the signal. The greater the number of turns in the auxiliary coil, the more sensitive it is to the noise in the environment. This results increase in the overall material cost.

The existing systems and/or methods of eliminating the RF interference in an MRI system involves additional component and material, which add on to the existing cost of the MRI system and at the same time, the overall weight of the MRI system and installation cost also increases.

Further limitations and disadvantages of conventional and traditional approaches will become apparent to one of skill in the art, through comparison of such systems with some aspects of the present disclosure as set forth in the remainder of the present application with reference to the drawings.

### BRIEF DESCRIPTION

This summary introduces concepts that are described in more detail in the detailed description. It should not be used to identify essential features of the claimed subject matter, nor to limit the scope of the claimed subject matter. Its sole purpose is to present concepts in a simplified form as a prelude to the more detailed description that is presented later.

In accordance with an aspect of the disclosure, a system may be provided to eliminate the radio frequency interference in a magnetic resonance imaging device. The system may comprise a plurality of coils configured to generate one or more data sets and a processor may be configured to receive the data sets from the plurality of coils. The processor may determine an interference removal parameter from the data sets received from the plurality of coils. The processor may remove a radio frequency interference, by the interference removal parameter, from the one or more data sets.

In accordance with an aspect of the disclosure, a method may be provided to eliminate the radio frequency interference in a magnetic resonance imaging device. The method may comprise determining a radio frequency interference at each coil. The method may comprise estimating an interference removal parameter and the method may, further, comprise designing and placing the interference removal parameter in form of a matrix in a reconstruction unit.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### DRAWINGS

The present invention will be better understood from reading the following description of non-limiting examples, with reference to the attached drawings, wherein below:

FIG. 1 illustrates an example for a magnetic resonance imaging (MRI) system, according to an aspect of the disclosure

FIG. 2 illustrates an imaging system, according to an aspect of the disclosure.

FIG. 3 illustrates the imaging system for noise elimination, according to an aspect of the disclosure.

FIG. 4 illustrates a method for an imaging system, according to an aspect of the disclosure.

FIG. 5 illustrates a method of estimating the interference removal parameter for a far-field system and a non-imaging data, according to an aspect of the disclosure.

FIG. 6 illustrates a method of estimating the interference removal parameter for a near-field system and a non-imaging data, according to an aspect of the disclosure.

FIG. 7 illustrates a method of estimating the interference removal parameter for a far-field system and an imaging data, according to an aspect of the disclosure.

FIG. 8 illustrates a method of estimating the interference removal parameter for a near-field system and an imaging data, according to an aspect of the disclosure.

FIG. 9 illustrates a method of estimating the interference removal parameter for a far-field system when the radio frequency interference may be injected during a non-imaging data acquisition, according to an aspect of the disclosure.

FIG. 10 illustrates a method of estimating the interference removal parameter for a near-field system when the radio frequency interference may be injected during a non-imaging data acquisition, according to an aspect of the disclosure.

The figures are not scale. Wherever possible, the same reference numbers will be used throughout the drawings and accompanying written description to refer to the same or like parts.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized, and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various examples of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

As used herein, the term “computer” and related terms, e.g., “computing device”, “computer system” “processor”, “controller” are not limited to integrated circuits referred to in the art as a computer, but broadly refers to at least one microcontroller, microcomputer, programmable logic controller (PLC), application specific integrated circuit, and other programmable circuits, and these terms are used interchangeably herein.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

As used herein, the terms “systems”, “devices” and “apparatuses are interchangeable and include components, sub-components, sub-systems that include without limitation the medical imaging devices.

The term “source model” or “a machine learning model” or a “machine learning module” is used herein to refer to an AI/ML model configured to perform a signal processing or analysis task on quasi-stationary signals. The signal processing or analysis task can vary. In various examples, the signal processing or analysis task can include, (but is not limited to): a segmentation task, an image reconstruction task, an object recognition task, a motion detection task, a video tracking task, an optical flow task, an attention region identification task, an object labeling task and the like. The source model can employ various types of AI/ML algorithms, including (but not limited to): deep learning models, neural network models, deep neural network models (DNNs), convolutional neural network models (CNNs), and the like. The terms “source domain model”, “source model” “source image processing model”, “source domain image processing model” and the like are used herein interchangeably to refer to an imaging processing model trained on images from specific domain, referred to herein as the source domain. Images included in the source domain are referred to herein as “source domain images” or “source images.” The terms “target domain model”, “target model”, “target image processing model”, “target domain image processing model”, and the like, are used herein interchangeably to refer to an imaging processing model configured to perform a same or similar image processing task as a corresponding source domain model, yet on images from a different but similar domain, referred to herein as the “target domain.” Images included in the target domain are referred to herein as “target domain images” or “target images”.

While certain examples are described below in the context of medical or healthcare systems, other examples can be implemented outside the medical environment.

In accordance with an aspect of the disclosure, a system may be provided to eliminate the radio frequency interference in a magnetic resonance imaging device. The system may comprise a plurality of coils configured to generate one or more data sets and a processor may be configured to receive the data sets from the plurality of coils. The processor may determine an interference removal parameter from the data sets received from the plurality of coils. The processor removes a radio frequency interference, by the interference removal parameter, from the one or more data sets.

In accordance with an aspect of the disclosure, a method may be provided to eliminate the radio frequency interference in a magnetic resonance imaging device. The method may comprise determining a radio frequency interference for each coil. The method may comprise estimating an interference removal parameter and the method may, further, comprise removing the radio frequency interference from the one or more data sets.

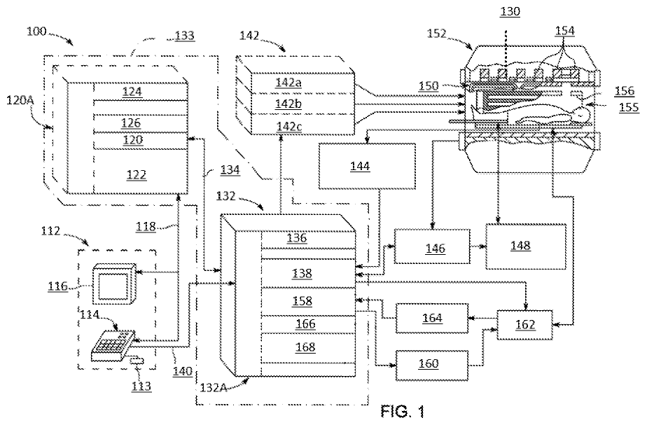

In accordance with an aspect of the disclosure, FIG. 1 illustrates an example for a magnetic resonance imaging (MRI) system 100. Operation of the magnetic resonance system 100 may be controlled from an operator console 112, which includes a keyboard and/or other input device 113, a control panel 114, and a display screen 116. The input device 113 can include a mouse, joystick, keyboard, track ball, touch activated screen, light wand, voice control, and/or other input device, and can be used for interactive geometry prescription, etc. The console 112 communicates through a link 118 with a computer system 120 that enables an operator to control the production and display of images on the display screen 116. The computer system 120 includes modules that communicate with each other through a backplane 120a. The modules of the computer system 120 comprises an image processor module 122, a central processing unit (CPU) module 124, and a memory module 126 that may comprise a frame buffer for storing image data arrays, for example. The computer system 120 may be connected to archival media devices, permanent or back-up memory storage or a network for storage of image data and programs and communicates with a separate MRI system control 132 through a high-speed signal link 134. The computer system 120 and the MRI system control 132 collectively form an “MRI controller” 133.

The MRI system control 132 comprises a set of modules connected together by a backplane 132a. These modules comprise a CPU module 136 as well as a pulse generator module 138. The CPU module 36 connects to the operator console 112 through a data link 140. The MRI system control 132 receives commands from the operator through the data link 140 to indicate the scan sequence that may be performed. The CPU module 136 operates the system components to carry out the desired scan sequence and produces data which indicates the timing, strength and shape of the RF pulses produced, and the timing and length of the data acquisition window. The CPU module 136 connects to several components that are operated by the MRI controller 133, including the pulse generator module 138 (which controls a gradient amplifier 142, further discussed below), a physiological acquisition controller (PAC) 144, and a scan room interface circuit 146.

The CPU module 136 receives patient data from the physiological acquisition controller 144, which receives signals from a number of different sensors connected to the patient, such as ECG signals from electrodes attached to the patient. The CPU module 136 receives, via the scan room interface circuit 146, signals from various sensors associated with the condition of the patient and the magnet system. The scan room interface circuit 146 also enables the MRI controller 133 to command a patient positioning system 148 to move the patient or client C to a desired position for the scan. The pulse generator module 138 operates the gradient amplifiers 142 to achieve desired timing and shape of the gradient pulses that are produced during the scan. The gradient waveforms produced by the pulse generator module 138 are applied to the gradient amplifier system 142 having Gx-142a, Gy-142b, and Gz-142c amplifiers. Each gradient amplifier excites a corresponding physical gradient coil in a gradient coil assembly, generally designated 150, to produce the magnetic field gradients used for spatially encoding acquired signals. The gradient coil assembly 150 forms part of a magnet assembly 152, which also comprises a polarizing magnet 154 (which, in operation, provides a homogeneous longitudinal magnetic field B0 throughout a target volume 155 that may be enclosed by the magnet assembly 152) and a whole-body RF (radio frequency) coil 156 (which, in operation, provides a transverse magnetic field B1 that may be generally perpendicular to B0 throughout the target volume 155). In certain examples, the RF coil 156 may be a multi-channel coil. A transceiver module 158 in the MRI system control 132 produces pulses that are amplified by an RF amplifier 160 and coupled to the RF coil 156 by a transmit/receive switch 162. The resulting signals emitted by the excited nuclei in the patient may be sensed by the same RF coil 156 and coupled through the transmit/receive switch 162 to a preamplifier 164. The amplified MR signals are demodulated, filtered, and digitized in the receiver section of the transceiver 158. The transmit/receive switch 162 may be controlled by a signal from the pulse generator module 132 to electrically connect the RF amplifier 160 to the coil 156 during the transmit mode and to connect the preamplifier 164 to the coil 156 during the receive mode. The transmit/receive switch 162 can also enable a separate RF coil (for example, a surface coil) to be used in either transmit mode or receive mode.

After the multi-channel RF coil 156 picks up the RF signals produced from excitation of the target, the transceiver module 158 digitizes these signals. Due to high sensitivity of the RF coil 156, the stray signals including noise signals may also get received by the RF coil 156. The source of stray signal can be either internal or external. There may be artificial noise source 130, which may generate artifacts in the data set. The MRI controller 133 then processes the digitized signals by Fourier transform to produce k-space data, which, then, may be transferred to a memory module 166, or other computer readable media, via the MRI system control 132. “Computer readable media” may include, for example, structures configured so that electrical, optical, or magnetic states may be fixed in a manner perceptible and reproducible by a conventional computer (e.g., text or images printed to paper or displayed on a screen, optical discs, or other optical storage media, “flash” memory, EEPROM, SDRAM, or other electrical storage media; floppy or other magnetic discs, magnetic tape, or other magnetic storage media).

A scan may be complete when an array of raw k-space data has been acquired in the computer readable media 166. This raw k-space data may be rearranged into separate k-space data arrays for each image to be reconstructed, and each of these k-space data arrays may be input to an array processor 168, which operates to Fourier transform the data into an array of image data. This image data may get conveyed through the data link 134 to the computer system 120, where it may be stored in memory. In response to commands received from the operator console 112, this image data may be archived in a long-term storage, or it may be further processed by the image processor 122 and conveyed to the operator console 112 and presented on the display 116.

In certain examples, the MRI controller 133 includes an example image quality (IQ) controller implemented using at least one of the CPU 124 and/or other processor of the computer system 120A and/or the CPU 36 and/or other processor of the system control 132A and/or a separate computing device in communication with the MRI controller 133.

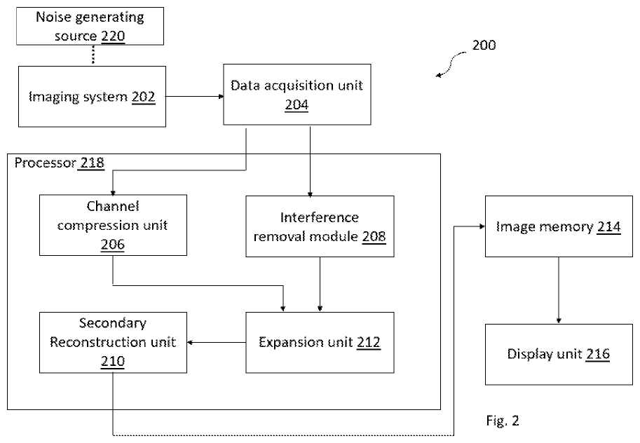

In accordance with an aspect of the disclosure, FIG. 2 illustrates a system 200 for removal of noise in an imaging system, which may comprise a Magnetic Resonance Imaging (MRI) system (for diagnostic/medical purpose, such as medical resonance imaging). The imaging system 202 may be comprising a plurality of radio frequency (henceforth referred to as RF) coils (not shown), which may be a primary RF coil. The primary coils may be configured to get induced by the magnetic resonance signal due to spin polarization of the atoms in the subject's body in response to an excitation pulse sequence. The plurality of RF coils may generate one or more data sets in response to the received magnetic resonance signal. In an example, the data set may comprise a non-imaging data or an imaging data. The imaging data and non-imaging data may comprise a magnetic resonance (henceforth referred to as MR) data and noise signal respectively. In an example, the source of noise signal may be an internal component of the MRI or an external noise generating source (220).

In accordance with an aspect of the disclosure, the system may comprise a processor 218, which may be connected to the plurality of RF coils, in an example. In another example, the processor 218 may be connected to the plurality of RF coils via a data acquisition unit 204. The data acquisition unit 204 may store the data set acquired from the plurality of coils either permanently or temporarily. In an example, the MR data may be obtained simultaneously along with the noise signal and in another example, the noise signal may be obtained without the MR data. Since, the data sets received from the RF coils may be weak signal, the data acquisition unit 204 may comprise an amplification circuit to amplify the data sets and a digital converter to convert the received data sets into a digital format. Once the data set may be amplified and converted into digital format, the processor 218 may receive the data set for processing.

In an example, the data acquisition unit 204 may be an integrated unit within the imaging system 202 and in another example, the data acquisition unit 204 may be an independent unit, which may be used for processing the data set, separately in other system, acquired from the imaging system 202. In another aspect of the disclosure, a processor 218 may be configured to process the data sets received from the plurality of RF coils of the imaging system 202. In an example, the RF coils are multi-channel primary RF coils and the processor 218 reconstructs signals from the multi-channel primary RF coils. Further, the processor 218 may comprise a channel compression unit 206. The channel compression unit 206 compresses or combines signals from some of the multi-channel RF primary coils or all the multi-channel primary coils to reduce the number of signals from various multi-channel primary RF coils to be reconstructed. The channel compression unit 206 reduces the processing time for the reconstruction of the channels. The processor 218, further comprises an interference removal module 208 which determines an interference removal parameter. The interference removal parameter may be determined from the data sets received from the plurality of RF coils. Once the interference removal parameter may be determined, a noise signal generated by the radio frequency interference may be eliminated. The interference removal parameter estimation may involve estimating the interference removal matrix which may perform weighted linear combination of the data from plurality of RF coils and remove or suppress the interference. Diagonal elements of the interference removal matrix may be set to zero to indicate that inference at a coil is estimated from weighted linear combination of all the other plurality of RF coils. The interference removal matrix may be estimated from the signal simultaneously received from the plurality of RF coils. In an example, s(t) may be a column vector wherein each element of the column vector may be the signal received simultaneously by the plurality of the RF coils. The value of t spans the duration over which the data may be captured continuously by the data acquisition unit 204. Fourier transform of s(t) along the t gives S(f) which indicate signal received at frequency f. The signal may consist of imaging signal, S^{Im}(f) and RF interference, S^{RF}(f), S(f)=S^{IM}(f)+S^{RF}(f). For RF interference matrix estimation, calibration data is used which has minimal to no imaging signal present. Calibration signal may consist multiple S(f) to estimate RF interference removal matrix R(f) such that, S^{RF}(f)=R(f)*S^{RF}(f) with diagonal elements of R(f) is zero. R(f) may be estimated by estimating each row of R(f) which corresponds to estimating the weights to be multiplied to all the other multitude of RF coils before linearly adding them to estimate the RF interference at a given coil. The RF interference may be removed/suppressed from the imaging signal by pre-multiplying the identity matrix minus the interference removal matrix.

$$\begin{matrix}( I - R ( f ) ) * S ( f ) = ( I - R ( f ) ) * ( {S}^{IM} ( f ) + {S}^{RF} ( f ) ) \\ = ( I - R ( f ) ) * {S}^{IM} ( f ) + ( I - R ( f ) ) * {S}^{RF} ( f ) \\ = ( I - R ( f ) ) * {S}^{IM} ( f ) + {S}^{RF} ( f ) + R ( f ) * {S}^{RF} ( f ) \\ = ( I - R ( f ) ) * {S}^{IM} ( f ) + {S}^{RF} ( f ) + {S}^{RF} ( f ) \\ = ( I - R ( f ) ) * {S}^{IM} ( f )\end{matrix}$$

The loss in imaging signal may recovered by further pre-multiplying with a pseudo-inverse matrix which may be Moore Penrose pseudo-inverse with Tikonov regularization denoted by (I−R(f))^{-p}. So noise inference removal with noise interference removal parameters may consist of pre-multiplication by (I−R(f))^{-p}*(I−R(f))^{-p}.

The interference removal module 208 may be configured to the data acquisition unit 204 and a expansion unit 212. The data acquisition unit 204 may comprise the data set, which may, further, comprise the noise signal. The undesirable noise may get generated due to the radio frequency interference from the internal components of the imaging system or any external source. The RF coils may be highly sensitive and may operate like an antenna thereby making it susceptible to receive the undesirable radio frequency interference signals causing noise pattern in the final images.

The interference removal module 208, having the interference removal parameter, may be configured to remove the noise signal. The interference removal parameter may be generated by using the data used for generating the images, referred to as imaging data or using the additional non-imaging data. In accordance with an aspect of the disclosure, either of the imaging data or the non-imaging data may enable capturing of a calibration data or a pre-calibration data, respectively. In accordance with an aspect of the disclosure, the calibrated data or the pre-calibrated data may be captured when the source of radio frequency interference produces either a near-field electromagnetic wave regions or a far-field electromagnetic wave regions, which may be formed by the noise generating source 220. RF interference due to near field may be frequency dependent while the RF interference due to far field may not be frequency dependent and RF interference removal can be further simplified.

In accordance with an aspect of the disclosure, when the data set may be the imaging data then the calibration data may be captured during multi-scan data acquisition. Multi-scan acquisition may improve the signal to noise ratio compared to single-scan acquisition. In accordance with an aspect of the disclosure, when the data set may be the non-imaging data, then pre-calibration data may be captured to determine the interference removal matrix. Once the interference removal matrix eliminates the noise signal from the data set, the noise free data set may be sent to an expansion unit 212.

In accordance with an aspect of the disclosure, the expansion unit 212 may expand the reduced number of channels from the channel compression unit 206 back to the original number of received channels. A secondary reconstruction unit 210 configured to receive the data sets from the expansion unit 212. The secondary reconstruction unit 210 reconstructs the expanded data into an image representation which may be stored in an image memory 214. The images stored in the image memory 214 may be displayed or represented on a display unit 216.

In accordance with an aspect of the disclosure, the noise may be extracted from the pre-calibrated data set or the calibrated data set, wherein the noise may be the result of the radio frequency interference caused due to a stray signal, which may get received by each of the plurality of RF coils of the imaging system 100 and radio frequency interference sources may be an oscillator, a passive component such as an inductor, a capacitor, and/or a transformer etc.

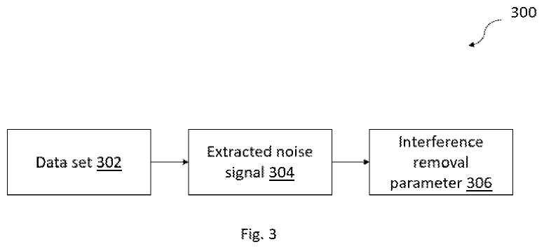

In accordance with an aspect of the disclosure, FIG. 3 illustrates the imaging system 300 for noise elimination. The magnetic resonance imaging (MRI) system 100 may be configured to generate a data set 302, which may be the imaging data set or a non-imaging data set, which may enable capturing of pre-calibrated data set or a calibrated data set, respectively. The RF coils of the imaging system 100 may be exposed to an external noise generating source (220), which may include, but not limited to, an oscillator, a passive component such as an inductor, a capacitor, and/or a transformer etc. The external noise generating source (220) may create a field system i.e., either a near-field system or a far-field system. Both the near field system as well as the far field system may create radio frequency (RF) interference, which may get received by the sensitive RF coils of the imaging system 100. To determine the interference removal parameter or the interference removal matrix for the far-field system, the RF interference source may be assigned same weight throughout all the frequencies of the RF coils. For the near-field system, each of the frequency may be assigned a unique, non-identical and independent weight for determining the interference removal parameter.

### Near Field/Transition Field RF Interference

The RF interference across coils are linearly related for a particular frequency,

Interferenceₚᵢ(fₖ)=Σ_{primary coil,j≠i}rᵢⱼₖ^{interference}^{ₚⱼ}^{(f}^{ₖ}⁾ (1)

### Far Field/transition field RF interference

The RF interference across coils are linearly related for a particular frequency,

Interferenceₚᵢ(fₖ)=Σ_{primary coil,j≠i}rᵢⱼₖ^{interference}^{ₚⱼ}^{(f}^{ₖ}⁾ (2)

Interferenceₚᵢ(t)=Σ_{primary coil,j≠i}rᵢⱼₖ^{interference}^{ₚⱼ}^{(t)} (3)

wherein, pᵢ represents an i^{th} primary RF coil;
pⱼ represents a j^{th} primary RF coil;
rᵢⱼₖ represents a function of the i^{th} primary RF coil and the j^{th} primary RF coil at a frequency (k) for the near-field system;
rᵢⱼ represents a function of primary RF coil (i) and primary RF coil (j) for far-field system; and
fₖ represents a frequency of a primary RF coil, wherein k representing the frequency index.

In accordance with an aspect of the disclosure, for near-field system, the radio frequency interference at the i^{th} primary RF coil (i) may be equal to a linear combination of the interferences at all the j^{th} primary RF coils (j), where j≠i, and rᵢⱼₖ may be the function of i and j and frequency (k). For far-field system the interference at the i^{th} primary RF coil (i) may be equal to a linear combination of all the interferences at the j^{th} primary RF coil (j), where j≠i. and rᵢⱼ may be only function of i and j and not frequency (k).

In accordance with an aspect of the disclosure, the noise signal may be indicative of the radio interference caused due to a signal received from the noise generating source (220). The noise signal 304 may be extracted from the data set 302 for each of the RF coils of the imaging system 100. The extracted noise signal 304 may be used to determine an interference removal parameter 306. The interference removal parameter may be a linear combination of the noise signal extracted from the data set. The noise signal may be extracted from a pre-calibrated data or a calibrated data. Pre-calibration data may be captured when the imaging system 100 may be operated without the presence of a subject inside the imaging system 100 and the calibration data may be generated when the subject may be kept within the imaging system for generating the imaging data.

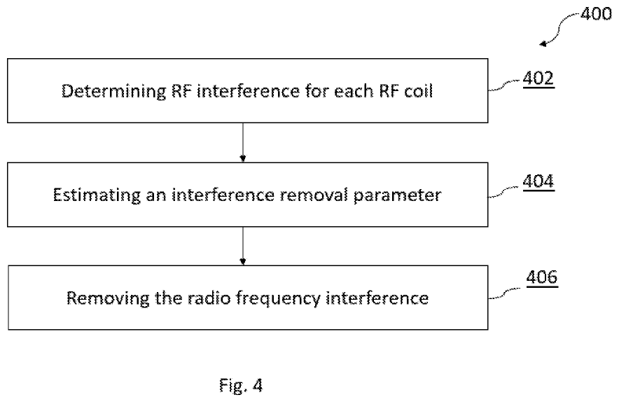

In accordance with an aspect of the disclosure, FIG. 4 illustrates a method 400 for an imaging system. In accordance with an aspect of the disclosure, the method may disclose determining a radio frequency (RF) interference 402 for each RF coil of the imaging system 100. The RF coils 100 may be susceptible to receive the extraneous noise signals. A noise data generated during reception of the noise signals may enable estimation of an interference removal parameter 404. In an example, the interference removal parameter may be in form of a matrix—an interference removal matrix. Once the interference removal parameter may be determined, the interference removal parameter may be implemented in other imaging system by incorporating the interference removal parameter in a reconstruction unit (not shown) of a processing device of the imaging system. In step 406, the radio frequency interference may be eliminated from the imaging data sets.

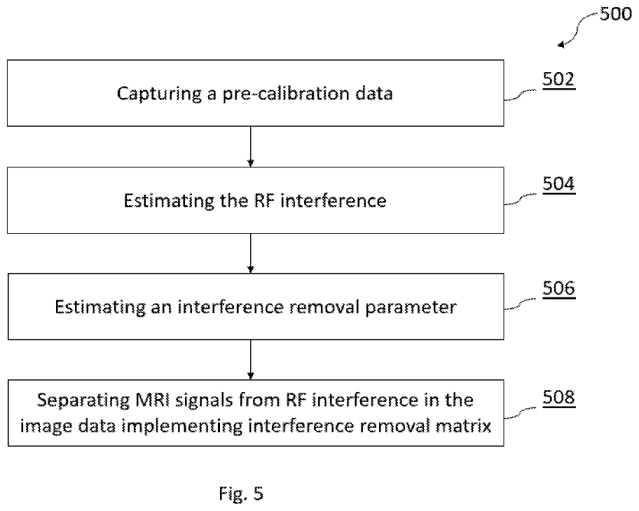

In accordance with an aspect of the disclosure, FIG. 5 illustrates a method 500 of estimating the interference removal parameter for a far-field system when the data set may comprise a non-imaging data. The process of estimating the interference removal parameter for the non-imaging data may be implemented prior to the initiation of the scanning process. Further, the disclosure may provide the method of estimating the interference removal parameter when a noise generating source (220) may be an external source or an internal source or both.

In an example, interference removal parameter may be estimated when the data set may be non-imaging data and the source of the radio frequency interference may generate far-field regions of electromagnetic waves i.e., a far-field system. In accordance with an aspect of the disclosure, the method may comprise capturing of a pre-calibration data 502. Once the pre-calibration data or the non-imaging data may be captured by the imaging system, the method may comprise, estimating the radio frequency interference for the far-field system, in step 504. The radio frequency interference at each RF coil of the imaging system 100 may be capable of generating the noise data, which may be captured, and an interference removal parameter may be determined from the linear combination of the interferences at the each of the RF coil, in step 506. In an example, the interference removal parameter may be a matrix calculated from the linear combination of the interferences at each of the coil of the imaging system. In step 508, the method may further comprise, separating a magnetic resonance imaging (MRI) signal from the RF interference in an image data by implementing the RF interference removal matrix during the final reconstruction process. In the far-field system, the radio frequency interference may remain same for all the entire channel of frequencies. In far-field system, a common weight may be assigned to each frequency and each of the frequency may be indicative of the coils of the imaging system. The image data may have one or more regions. Some of the regions may have projection of the anatomy of the subject and rest of the remaining regions may have only the artifact or noise. The region with the artifacts or noise may be used to determine the radio frequency interference matrix.

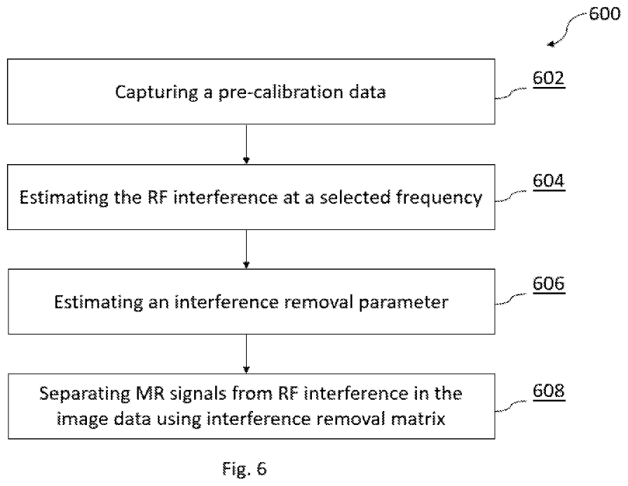

In accordance with an aspect of the disclosure, FIG. 6 illustrates a method 600 of estimating the interference removal parameter for a near-field system when the data set may comprise a non-imaging data. In another example, interference removal parameter may be estimated when the data set may be non-imaging data and the external source of the radio frequency interference may generate near-field regions of electromagnetic waves, a near-field system. In accordance with an aspect of the disclosure, the method may comprise capturing of a pre-calibration data 602. Once the pre-calibration data or the non-imaging data may be captured by the imaging system, the method may comprise, estimating the radio frequency interference for the near-field system, in step 604. The radio frequency interference may be determined at a selected frequency. In the near-field system, the radio frequency interference varies from one frequency to another unlike the far-field system. In near-field system, a weight may be assigned to each of the frequency. The weight, determined by the pre-scan data and the scan data, assigned to a particular frequency may be unique and independent from the weight assigned to other frequencies. In an example, the interference removal parameter may be a matrix estimated, step 606, from the linear commination of the interferences at each of the coil of the imaging system. In step 608, the method may further comprise, separating a magnetic resonance imaging (MRI) signal from the RF interference in an image data by implementing the RF interference removal matrix during the final reconstruction process.

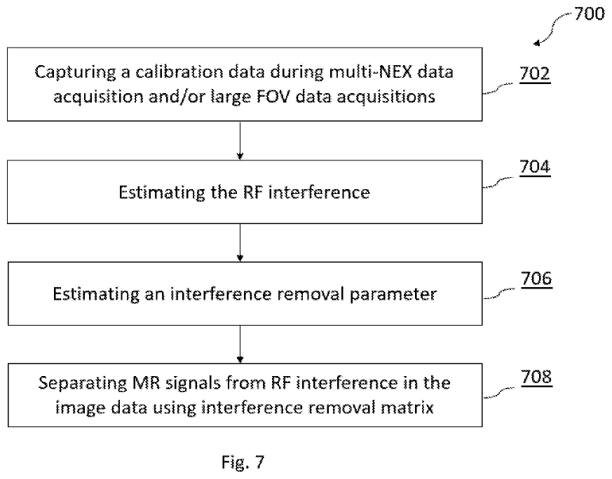

In accordance with an aspect of the disclosure, FIG. 7 illustrates a method 700 of estimating the interference removal parameter for a far-field system when the data set may comprise an imaging data. In accordance with an aspect of the disclosure, the method may comprise capturing a calibration data during multi-scan data acquisition and/or field of view data acquisition, in step 702.

In an example, the noise signal may be extracted from the multi-scan data acquisition by subtracting a subsequently acquired scanned signal from the previously acquired scanned signal, wherein the subsequently acquired scanned signal and previously acquired scanned signal are captured one after the another. Further, the method may comprise, estimating the radio frequency interference for all the frequency indicative of noise received by the RF coils of the imaging system 100, in step 704. In step 706, the method may comprise, estimating the interference removal parameter in form of interference removal matrix, which may be a linear combination of the interferences at each of the RF coil of the imaging system 100. The method may further comprise, separating a magnetic resonance imaging (MRI) signal from the RF interference in an image data by implementing the RF interference removal matrix during the reconstruction process, in step 708. In the far-field system, the radio frequency interference may remain same for the entire channel of frequencies and same weight may be assigned to each frequency to determine the interference removal matrix.

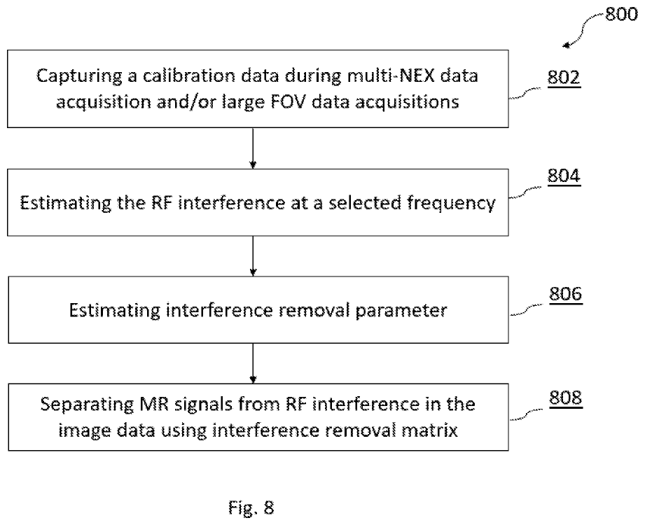

In accordance with an aspect of the disclosure, FIG. 8 illustrates a method 800 of estimating the interference removal parameter for a near-field system when the data set may comprise an imaging data. In accordance with an aspect of the disclosure, the method may comprise capturing a calibration data during multi-scan data acquisition and/or field of view data acquisition, in step 802. Further, the method may comprise, estimating the radio frequency interference for all the frequency, individually, indicative of each of the coil of the imaging system, in step 804. In step 806, the method may comprise, estimating the interference removal parameter in form of interference removal matrix, which may be a linear combination of the interferences at each of the coil of the imaging system. The method may further comprise, separating a magnetic resonance imaging (MRI) signal from the RF interference in an image data by implementing the RF interference removal matrix during the reconstruction process, in step 808.

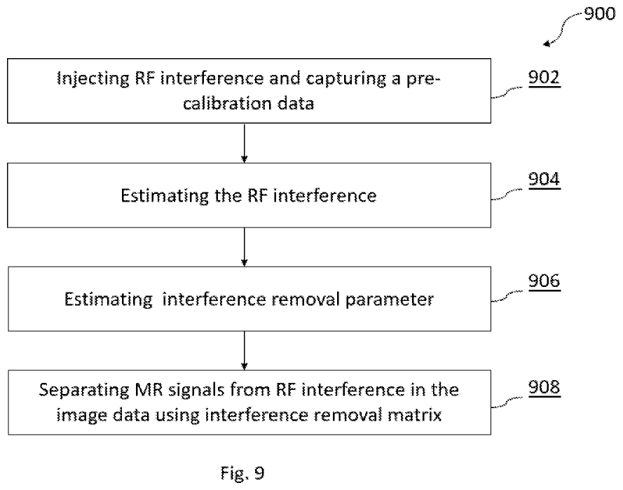

In accordance with an aspect of the disclosure, FIG. 9 illustrates a method 900 of estimating the interference removal parameter for a far-field system when the radio frequency interference may be injected during a non-imaging data acquisition. The method may comprise, step 902, injecting radio frequency interference to create an artificial radio frequency interference and capturing a pre-calibration data. Further, the method may comprise, estimating the RF interference, in step 904. Once, the radio frequency interference may be estimated, the interference removal parameter may be estimated in step 906. The method may further comprise, separating a magnetic resonance imaging (MRI) signal from the RF interference in the image data by implementing the interference removal matrix during the reconstruction process, in step 908. The radio frequency interference may be injected in a coil when anticipation of the interference may be uncertain or may not be known. The radio frequency interference may occur at any stage of operation of the imaging system 100. The radio frequency interference may be injected using a noise generating source (220) by operating the noise generating source (220) at a frequency equivalent to the frequency at which at least one of the RF coils of the imaging system 100 may be operating.

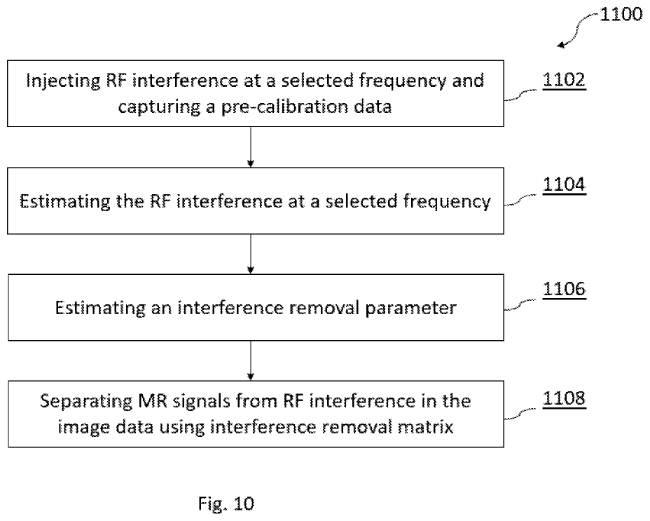

In accordance with an aspect of the disclosure, FIG. 10 illustrates a method 1100 of estimating the interference removal parameter for a near-field system when the radio frequency interference may be injected during a non-imaging data acquisition. The method may comprise, step 1102, injecting radio frequency interference at a selected frequency and capturing a pre-calibration data at the selected frequency. Further, the method may comprise, estimating the RF interference at the selected frequency, in step 1104. Once, the radio frequency interference may be estimated, the interference removal parameter may be estimated in step 1106. The method may further comprise, separating a magnetic resonance imaging (MRI) signal from the RF interference in the image data by implementing the interference removal matrix during the reconstruction process, in step 1108.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but may not be limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In accordance with an aspect of the disclosure, respective components can execute from various computer readable media having various data structures stored thereon. The components can communicate via local and/or remote processes such as in accordance with a signal having one or more data packets/data sets (e.g., data from one component interacting with another component in a local system, distributed system, and/or across a network such as the Internet with other systems via the signal). In accordance with another aspect of the disclosure, a component can be an apparatus with specific functionality provided by mechanical parts operated by electric or electronic circuitry, which may be operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In accordance with another aspect of the disclosure, a component can emulate an electronic component via a virtual machine, e.g., within a cloud computing system.

In addition, the term “or” may be intended to mean an inclusive “or” rather than an exclusive “or.” That is, unless specified otherwise, or clear from context, “X employs A or B” may be intended to mean any of the natural inclusive permutations. That is, if X employs A; X employs B; or X employs both A and B, then “X employs A or B” may be satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein may not be limited by such examples. In addition, any aspect or design described herein as an “example” and/or “exemplary” may not necessarily to be construed as preferred or advantageous over other aspects or designs, nor it may be meant to preclude equivalent exemplary structures and techniques known to those of ordinary skill in the art.

As it may be employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or non-volatile memory or can include both volatile and non-volatile memory. By way of illustration, and not limitation, non-volatile memory can include read only memory (ROM), programmable ROM (PROM), electrically programmable ROM (EPROM), electrically erasable ROM (EEPROM), flash memory, or non-volatile random-access memory (RAM) (e.g., ferroelectric RAM (FeRAM). Volatile memory can include RAM, which can act as external cache memory, for example. By way of illustration and not limitation, RAM may be available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” may be interpreted when employed as a transitional word in a claim. The descriptions of the various examples have been presented for purposes of illustration, but are not intended to be exhaustive or limited to the examples disclosed. Many modifications and variations can be apparent to those of ordinary skill in the art without departing from the scope and spirit of the described examples. The terminology used herein was chosen to best explain the principles of the examples, the practical application or technical improvement over technologies found in the marketplace, or to enable others of ordinary skill in the art to understand the examples disclosed herein.

This written description uses examples to disclose the invention, including the best mode, and to enable any person skilled in the art to practice the invention, including making and using any computing system or systems and performing any incorporated methods. The patentable scope of the invention may be defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Examples of the present disclosure shown in the drawings and described above are example examples only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described examples can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. An imaging system comprising:
a plurality of coils configured to generate one or more data sets;
a processor connected to the plurality of coils, wherein the processor is configured to:
receive the one or more data sets from the plurality of coils;
determine an interference removal parameter from the one or more data sets received from the plurality of coils; and
remove a radio frequency interference, by the interference removal parameter, from the one or more data sets.

2. The imaging system of claim 1, wherein the plurality of coils are multi-channel radio frequency (RF) coils, and wherein the multi-channel RF coils are primary RF coils.

3. The imaging system of claim 1, wherein the plurality of coils are exposed to a field system and generate one or more data sets, wherein the field system is a near-field system or a far-field system.

4. The imaging system of claim 1, wherein the one or more data sets comprises an imaging data and a non-imaging data, and wherein a calibrated data set is captured from the imaging data and the pre-calibrated data is captured from the non-imaging data.

5. The imaging system of claim 4, wherein a noise data is extracted from the pre-calibrated data set and the calibrated data set to estimate the interference removal parameter, wherein the noise data is generated from the noise generating source.

6. The imaging system of claim 1, wherein the interference removal parameter is an interference removal matrix.

7. The imaging system of claim 6, wherein the interference removal matrix is a linear combination of the noise data extracted from the pre-calibrated data and the calibrated data.

8. The imaging system of claim 6, wherein the interference removal parameter is provided in an interference removal module, wherein the interference removal module is configured to send data set to a reconstruction unit and a data correction unit.

9. The imaging system of claim 7, wherein the linear combination of the noise signal for the near-field comprises computation of weights for each frequency independently indicative of each of the plurality of coils.

10. The imaging system of claim 7, wherein the linear combination of the noise signal for the far-field comprises computation of one set of equal weights for all the frequency indicative of the plurality of coils.

11. The imaging system of claim 1, wherein the one or more data sets comprise a field of view data acquired during a scanning process to estimate the interference removal parameter, and wherein the field of view data is acquired from an imaging data.

12. The imaging system of claim 1, wherein the interference removal matrix is a linear combination of the noise signal extracted from a portion of the scanned image without a projection of an anatomy of a subject.

13. The imaging system of claim 1, wherein the interference removal matrix is configured to a data correction and an expansion unit, wherein the data correction and the expansion unit enables the implementation of radio frequency interference removal matrix to extract the noise and the radio frequency interference from the one or more data sets.

14. The imaging system of claim 1, wherein an output from the data correction and the expansion unit is transferred to a reconstruction unit to produce a noise free image data; and the reconstructed image is stored in an image memory of the magnetic resonance imaging system.

15. A method comprising:
determining a radio frequency interference for each Radio Frequency coil;
estimating an interference removal parameter; and
removing the radio frequency interference from the one or more data sets.

16. The method of claim 15, wherein the determining the radio frequency interference for a radio frequency coil comprises assigning a unique and non-identical weight to each of the frequency for a near-field system.

17. The method of claim 15, wherein the determining the radio frequency interference for a coil comprises assigning a common weight to all the frequencies for a far-field system.

18. The method of claim 15, wherein the estimating the interference removal parameter comprises:
capturing a pre-calibration data to estimate interference removal matrix;
estimating the radio frequency interference at a selected frequency for the near-field system and estimating the radio frequency interference common to all frequencies for a far field system;
estimating an interference removal matrix; and
separating a Magnetic Resonance signal from the radio frequency interference in an image data using the Radio Frequency interference removal matrix.

19. The method of claim 15, wherein the estimating the interference removal parameter comprises:
capturing a calibration data during multi-scanning data acquisition or during field of view acquisition to estimate interference removal matrix;
estimating the radio frequency interference at a selected frequency for the near-field system and estimating the radio frequency interference common to all frequencies for the far-field system;
estimating a Radio frequency interference removal matrix; and
separating Magnetic resonance signals from radio frequency interference in an image data using the interference removal matrix.

20. The method of claim 15, wherein the estimating the interference removal parameter comprises:
injecting a radio frequency interference at a selected frequency;
capturing a pre-calibration data to estimate interference removal matrix;
estimating the radio frequency interference at the selected frequency for the near-field system;
estimating an Radio Frequency interference removal matrix; and
separating Magnetic Resonance signals from radio frequency interference in an image data using the interference removal matrix.

21. The method of claim 15, wherein the estimating the interference removal parameter comprises:
injecting a radio frequency interference;
capturing a pre-calibration data to estimate interference removal matrix;
estimating the radio frequency interference for the far-field system;
estimating an Radio Frequency interference removal matrix; and
separating Magnetic Resonance signals from radio frequency interference in an image data using the interference removal matrix.
